(19) United States
(12) Patent Application Publication
(10) Pub. No.: US 2001/0056068 A1
(43) Pub. Date: Dec. 27, 2001

(54) METHOD OF TREATMENT AND PREVENTION OF NITRIC OXIDE DEFICIENCY-RELATED DISORDERS WITH CITRULLINE AND CITRULLINE DERIVATIVES

(76) Inventors: KRISTOF CHWALISZ, BERLIN (DE); ROBERT E. GARFIELD, FRIENDSWOOD, TX (US); SHAO-QUING SHI, GALVESTON, TX (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/034,351

(22) Filed: Mar. 4, 1998

(51) Int. Cl. A61K 38/00; A61K 31/47

(52) U.S. Cl. 514/21

(57) ABSTRACT

The invention provides methods for control, management, treatment and prevention of conditions related to nitric oxide deficiency such as hypertension, cardiovascular disease, osteoporosis, diabetes mellitus, preeclampsia HELLP, syndrome and fetal growth retardation; uterine contractility disorders such as preterm labor and dysmenorrhea, cervical dystocia, infertility and early pregnancy loss; male impotence; urinary incontinence; intestinal tract disorders (e.g. altered motility and pyloric stenosis), respiratory system diseases (e.g. asthma, neonatal respiratory distress syndrome, pulmonary hypertension, and adult respiratory distress syndrome); inflammatory diseases (e.g. acute inflammation, resistance to infection, SLE-lupus, anaphylactic reaction, allograft rejection); Alzheimer's disease, stroke, growth hormone disorders, and behavior changes; dermatological conditions such as atopic eczema, topical hair loss, and burn injury; by administering citrulline or a citrulline analogue, optionally in combination with other enhancing or modulating agents, e.g., an estrogenic, partial estrogenic, progestagenic, or androgenic agent, and pharmaceutical preparations for such uses.

### BACKGROUND OF THE INVENTION

1. Field of the Invention

This invention concerns a method and agents for control, management, treatment and prevention of disorders and diseases related to nitric oxide deficiency or those disorders or diseases which can be improved by enhancing endogenous nitric oxide synthesis by providing to a mammal citrulline or a citrulline analogue alone or in combination with other enhancing or modulating agent. This invention relates to nitric oxide dependent disorders and diseases including, hypertension, cardiovascular disease, atherosclerosis, myocardial ischemia, preeclampsia, HELLP (severe preeclampsia (Hemolysis+Elevated Liver enzymes+Low Platelets) syndrome), and fetal growth retardation, osteoporosis, uterine contractility disorders, such as preterm labor and dysmenorrhea, cervical dystocia, male impotence, urinary incontinence, renal arterial stenosis. In addition, this invention relates to a method and agents for treatment of infertility by improving implantation rates or controlling ovulation. Furthermore, this invention relates to a method and agents for hormone replacement therapy (HRT) alone or in combination with steroid hormones or other enhancing or modulating agents in females during the menopause to prevent climacteric disorders such as hot flushes, abnormal clotting patterns, urogenital discomfort, increased incidence of cardiovascular diseases, etc., associated with the reduction in ovarian function in middle-aged women. This invention also concerns a method and agents for HRT alone or in combination with steroid hormones or other enhancing or modulating agents in males to prevent cardiovascular disease, osteoporosis and impotence.

There are also other potential uses of citrulline or citrulline analogues in those clinical situations in which nitric oxide plays a modulatory role. For example (1) regarding the cardiovascular system: regulation of vascular conductance, regulation of blood flow, regulation of blood pressure, (2) regarding the gastrointestinal tract and pancreas pathology: altered motility, pyloric stenosis, diabetes mellitus, (3) regarding respiratory system: asthma, treatment of premature babies to increase lung function (neonatal respiratory distress syndrome), pulmonary hypertension, adult respiratory distress syndrome, (4) in inflammation: autoimmune and immune diseases, acute inflammation, resistance to infection, SLE-lupus, anaphylactic reaction, allograft rejection, within the central nervous system: Alzheimer's disease, stroke, growth hormone disorders, behavior changes, (5) in dermatological conditions: atopic eczema, topical hair loss, and burn injury.

2. Background and Related Disclosures

One of the most exciting recent advances in biology and medicine is the discovery that the diffusible molecule nitric oxide is produced by endothelial cells and that it is involved in the regulation of vascular tone, plate-let aggregation, peripheral nitrergic transmission at smooth muscle, intra-cellular communication in the CNS, and macrophage defense mechanisms following exposure to bacterial products (Furchgott R F, Zawadzki J V. The obligatory role of endothelial cells in the relaxation of arterial smooth muscle by acetycholine. Nature 1980; 288:373-376.; Moncada S and Higgs E A. The L-arginine-nitric oxide pathway. New Engl J Med 1993; 329:2002-2012).

Nitric oxide is an important mediator of relaxation of the muscular smooth muscle, and was formerly known as EDRF (endothelin-derived relaxing factor). Nitric oxide elevates levels of the secondary mediator cGMP (1,3,5-cyclic guanosine mono-phosphate) within the vascular smooth muscle to produce relaxation and to reduce blood vessels tone. Nitric oxide binds to heme and in turn activates soluble guanylate cyclase to increase the cellular content of cGMP. Guanylate cyclase represents, therefore, the effector system for nitric oxide in the majority of tissues, including vascular and uterine smooth muscles, neurons, fibroblasts, platelets, etc. However, nitric oxide can also act by other mechanisms which are not cGMP-depen-dent (Ignarro L G. Biological actions and properties of endothelium-derived nitric oxide formed and released from artery and vein. Circulation Research 1989, 65:1-21; Moncada S and Higgs E A. The L-arginine-nitric oxide pathway. New Engl J Med 1993; 329:2002-2012).

Nitric oxide is synthesized by a family of nitric oxide synthases (NOS) which are enzymes that convert the amino acid L-arginine to citrulline and nitric oxide.

Three highly related NOS enzymes have been isolated and identified. These include endothelial NOS (ec-NOS, type III), neuronal NOS (nc-NOS, b-NOS, type I) and inducible NOS (i-NOS, type II) (Nathan C and Qia-wen Xie. Nitric oxide synthases: Roles, tolls, and controls. Cell 1994; 78:915-918). The constitutive isoforms ec-NOS and nc-NOS were originally identified in endothelial and neuronal tissues, respectively; they rapidly and transiently produce small amounts of NO under basal conditions.

The e-NOS form of the enzyme is expressed in endothelial cells, in cardiac myocytes, platelets and in some neurons. The ec-NOS-derived NO is the most important vasodilator. It is released gradually at low levels to maintain a constant vasorelaxation and normal blood pressure. Vasodilatation can be produced by vasoactive agents such as acetylcholine, and bradykinin acting in such a manner as to increase cytosolic Ca⁺⁺ levels in endothelial cells, thereby increasing ec-NOS activity and consequently raising nitric oxide production. In addition, steroid hormones and sheer stress can also increase ec-NOS activity (see below). Other agents can decrease ec-NOS activity and produce a vasoconstriction. Deficiencies or abnormally low activity of ec-NOS results in drastic alteration of cardiovascular function.

The nc-NOS isoform was the first isoform to be isolated and studied at the molecular level (Nathan C and Qia-wen Xie. Nitric oxide synthases: Roles, tolls, and controls. Cell 1994; 78:915-918). It is found in neuronal tissues of the central and peripheral nervous system and it is thought to act as a neurotransmitter. The i-NOS isoform is inducible by cytokines or endotoxin and produces large quantities of nitric oxide for hours or days in a Ca⁺⁺-independent manner.

The macrophage-type isoform i-NOS consistently produces nitric oxide when it is present in cells. It is induced (upregulated) by some cytokines (IL-1, IFN-γ, TNF-α) and inhibited by others (IL-4, IL-10, TGF-β). The expression of i-NOS is also enhanced by endotoxins such as lipopolysaccharide (LPS). i-NOS was first identified in immune cells (macrophages), but has now also been found in epithelial cells, hepatocytes, myocytes, fibroblasts, chondrocytes and bone-forming cells (osteoblasts and osteoclasts). Large quantities of i-NOS-derived nitric oxide can kill parasites and bacteria and it has been postulated that this pathway is the most significant in controlling the invasion of pathogens. This system also plays a pivotal role in tissue remodeling during acute and chronic inflammation.

The NOS isoforms are also expressed in the reproductive tract. The expression of NOS enzymes in the rat uterus was studied with immunoblotting with monoclonal antibodies. i-NOS and ec-NOS were detected in the uterus (myometrium). i-NOS, which represents the major NOS isoform in the uterus and cervix, is gestationally regulated. The uterine i-NOS enzyme decreased in the uterus during labor at term and preterm in animals treated to deliver prematurely. Opposite changes were observed in the cervix (Buhimschi I, Ali M, Jain V, Chwalisz K and Garfield R E. [1996], Differential regulation of nitric oxide in the uterus and cervix during pregnancy and labor. Human Reprod 11:1755-1766).

NOS is also present in placental tissues and uterine arteries. The trophoblast invasion of uteroplacental arteries in relation to the nitric oxide synthase isoform expression was studied in pregnant guinea pigs by means of immune- and histochemistry as compared to arterial dilatation. A pronounced dilatation of uteroplacental arteries begins at mid-pregnancy and progresses until term (Nanaev A, Chwalisz K, Frank H-G, Kohnen G, Hartung C-H and Kaufmann P. [1995], Physiological dilation of uteroplacental arteries in the guinea pig depends upon nitric oxide synthase activity of extravillous trophoblast. Cell Tissue Res:282: 407-421). This study demonstrates that dilatation of uteroplacental arteries can be seen when invading trophoblast cells coexpressing endothelial (ecNOS) and macrophage (iNOS) nitric oxide synthase are found in the vicinity of the vessels, i.e., prior to trophoblast invasion of the arterial walls.

Conrad et al.,(1993), localized NOS to the syncythiotrophoblast cell layer in human placenta (Conrad K P, Vill M, Mcguire P G, Dail W G, Davis A K [1993], Expression of nitric oxide synthase by syncythiotrophoblast in human placental villi, FASEB J 7:1269-1276). Morris et al., (1993), demonstrated both calcium-dependent and calcium-independent activity in human placental villi and the basal plate (Morris N H, Sooranna S R, Eaton B M, Steer P J (1993) NO synthase activity in placental bed and tissues from normotensive pregnant women. Lancet 342:679-680), and Myatt et al (1993), showed that placental villous tree synthesized a calcium-dependent-isoform of the NOS (Myatt L, Brockman D E, Langdon G, Pollock J S [1993], Constitutive calcium-dependent isoform of nitric oxide synthase in the human placenta villous vascular tree. Placenta 14:373-383; Myatt L, Brockman D E, Eis A L, Pollock J S [1993] Immunohistochemical localization of nitric oxide synthase in the human placenta. Placenta 14: 487-495). In addition, Buttery et al., (1994) showed that endothelial NOS at term was localized in the endothelium of umbilical artery and vein and in the placental syncythiotrophoblast (Buttery L D K, McCarthy A, Springall A et al., [1994], Endothelial nitric oxide synthase in the human placenta: regional distribution and proposed regulatory role at feto-maternal interface. Placenta 15: 257-267). Furthermore, Moorhead et al., (1995) have shown that NADPH diaphorase (non-specific reaction to identify nitric oxide synthase) was in various uterine components during early pregnancy (Moorhead C S, Lawhun M, Nieder G L [1995], Localization of NADPH diaphorase in the mouse uterus during the first half of pregnancy and during an artificially-induced decidual cell reaction. J Histochem Cytochem 43:1053-1060). Finally, Toth et al., (1995) demonstrated that NOS activity was present in the first trimester human placental homogenates (Toth M, Kukor Z, Romero R, Hertelendy F [1995], Nitric oxide synthase in first trimester human placenta: Characterization and subcellular distribution. Hypertens Pregnancy 14/3: 287-300). iNOS is also expressed in the implantation site (Purcell T L., Buhimschi I, Chwalisz K., Given R. and Garfield R. E. (1997) Spatiotemporal distribution of nitric oxide synthase (NOS) isoforms in the mouse implantation Site. 44th Annual Meeting of Society for Gynecologic Investigation, Abstract#640, San Diego, Calif., The San Diego Marriott, Mar. 19-22, 1997). In conclusion, these studies demonstrate that nitric oxide is synthesized in the uterus and placenta and represents an important factor regulating placental blood flow and myometrial quiescence during pregnancy. In addition, it plays an important role during implantation and decidual reaction.

The nitric oxide pathway can be regulated in a variety of ways by cytokines, steroids, prostaglandins, and other endogenous agents. The sex steroid hormones estradiol, progesterone and testosterone can modulate the NOS enzymes, guanylate cyclase and/or the effects of cGMP in the uterus and cervix, and other steroid hormone-dependent tissues such as blood vessels and bones. Steroid hormones modulate NOS expression, guanylate cyclase activity or the nitric oxide effector system (cGMP-dependent relaxation mechanism) in a tissue-specific manner. In the uterus, progesterone seems to be the primary hormone responsible for the up-regulation of nitric oxide during pregnancy (Chwalisz K, Buhimschi I, Garfield R E (1996) Role of nitric oxide in obstetrics. Prenat Neonat Med 1,: 292-329). On the other hand, progesterone down-regulates nitric oxide production in the uterine cervix. Estradiol is also believed to be responsible for the up-regulation of ecNOS and ncNOS in the endothelium and brain during pregnancy (Weiner C P, Lizasoain I, Baylis S A, Knowles R C, Charls I C, Moncada S. Induction of calcium-dependent nitric oxide synthases by sex hormones. Proc Natl Acad Sci USA 1994; 91:5212-16; Weiner C P, Knowles, R G, Moncada, S. Induction of nitric oxide synthases early in pregnancy. Am J Obstet Gynecol 1994; 171, 838-843.).

It has long been recognized that nitrovasodilators, such as nitroglycerin and sodium nitroprusside (SNP), inhibit vascular smooth muscle contractility to produce vascular relaxation and to reduce vascular tone. These agents have been used since the late 1800s as vasodilators. However, only within the last few years have the mechanisms of action of these substances become known (Moncada S and Higgs E A. The L-arginine-nitric oxide pathway. New Engl J Med 1993; 329:2002-2012). We now know that these compounds release nitric oxide (acting as nitric oxide donors) either spontaneously (e.g. SNP) or after metabolic conversion (e.g. nitroglycerin). Endogenous nitric oxide levels can also be raised by L-arginine (nitric oxide substrate) treatment. Since nitric oxide is involved in numerous pathophysiological processes, it is theoretically possible to overcome some of the previously mentioned health problems with NO donors. At present, these agents are mostly nonspecific, developing rapid tolerance, thus limiting their use. In future, NO-donors such as nitroglycerin will be replaced by new compounds that are more selective and lack the problems of tolerance. Nitric oxide synthesis by NOS enzymes has been shown to be competitively inhibited by numerous analogues of L-arginine including N^{G}-nitro-L-arginine methyl ester (L-NAME), N^{G}-monoethyl-L-arginine, monoacetate (L-NMMA), L-N⁵(-1-Iminoethyl)ornithine, hydrochloride (L-NIO, HCl), N^{G}-nitro-L-arginine (L-NNA), etc. A multitude of studies demonstrate that the inhibition of nitric oxide synthesis with these compounds results in a prolonged elevation in blood pressure in a variety of animal species (Furchgott R F, Zawadzki J V. The obligatory role of endothelial cells in the relaxation of arterial smooth muscle by acetycholine. Nature 1980; 288:373-376; Moncada S and Higgs E A. The L-arginine-nitric oxide pathway. New Engl J Med 1993; 329:2002-2012; Ignarro L G. Biological actions and properties of endothelium-derived nitric oxide formed and released from artery and vein. Circulation Research 1989; 65:1-21). These compounds are non-specific, i.e. they inhibit all NOS isoforms. Aminoguanidine and L-NIL are, however, specific to i-NOS. NOS inhibitors can be used in vivo to mimic or induce nitric oxide deficiency conditions. Nitric oxide deficiency conditions: There is a substantial body of evidence from animal experiments and human studies that a deficiency in nitric oxide contributes to the pathogenesis of a number of diseases of the cardiovascular system, including hypertension and the cardiovascular disease (e.g. atherosclerosis, restenosis). The inhibition of NOS with L-NNMA, L-NA or L-NAME dramatically increases blood pressure and promotes the development of atherosclerosis in laboratory animals. Based on our studies of the interactions on nitric oxide with steroid hormones, we proposed that various problems of women's reproductive health, including some pregnancy-related disorders, can be explained by nitric oxide deficiency (Chwalisz et al., 1996). During pregnancy, NO deficiency may be the underlying mechanism of various pathological conditions such as preeclampsia, preterm birth, cervical incompetence, recurrent abortions. During the reproductive age, nitric oxide deficiency may play a pivotal role in dysmenorrhea and infertility. The patophysiological conditions occurring during and after the menopause, including hot flushes, cardiovascular disease, urinary incontinence, cognition problems, etc., can also be related, at least in part, to nitric oxide deficiency. Similarly, in aging men the increased frequency of the cardiovascular disease, hypertension, impotence and osteoporosis may also be related to nitric oxide deficiency.

Epidemiological data indicate that approximately one half of deaths in economically developed countries are attributable to a cardiovascular disease including coronary heart disease, stroke, restenosis and other forms of vascular disease. The commonest and most lethal form of cardiovascular disease is coronary heart disease. In men, there is a continuous increase in the prevalence of cardiovascular disease after the age of 30-40 years. On the other hand, the rate of cardiovascular disease, especially coronary heart disease, is relatively low among premenopausal women, but rises after menopause suggesting that sex steroids (estrogens and progesterone) have a protective effect in women. Moreover, an increased prevalence of coro-nary heart disease was repeatedly reported in women after bilateral oophorectomy (Green A and Bain C, (1993) Epidemiological overview of estrogen replacement and cardiovascular disease. Baillière's Clinical Endocrinology and Metabolism 7:95-113).

The effects of sex steroids on the vessels is mediated by various locally produced hormones including nitric oxide, prostacylin and endothelins. There is growing evidence that nitric oxide plays an important role in the pathogenesis of atherosclerosis (Naito M, Hayashi T, and Iguchi A (1995) New approaches to prevention of atherosclerosis. Drugs 50: 440-453). There is an impairment of endothelium-dependent vasodilatation in humans and animals with hypercholesterolemia-induced atherosclerosis. In cholesterolemic rabbits, chronic inhibition of NOS accelerates atherogenesis and neointima formation, and increases endothelial adhesiveness to monocyte cells (Cayatte A J, Palocino J J, Horten K et al., (1994) Chronic inhibition of nitric oxide production accelerates neointima formation and impairs endothelial function in hypercholesterolemic rabbits. Arterioscl Thromb 14: 753-759). In conclusion, the steroid hormones estrogens, androgens and progesterone can modulate the nitric oxide production in a variety of steroid-hormone-dependent tissues.

Bone-remodeling disorders such as osteoporosis and osteoarthritis are frequently associated with perturbations in the interactions between local and systemic bone-remodeling regulatory pathways. Postmenopausal bone loss associated with diminished steroid hormones is correlated with increased levels of cytokines. In addition, both estrogen and progestins are effective in preventing postmenopausal bone loss (Abdalla H, Hart D M, Lindsey R, Leggate I, Hooke A. [1985] Prevention of bone mineral loss in postmenopausal women by norethisterone. Obstet Gynecol 66:789-792; Orwell E E and Klein R F. [1995] Osteoporosis and men. Endocrine Rev. 16:87-116). Bone-degrading osteoclasts arise from cells within the monocyte macrophage lineage. Excessive osteoclast activity leads to high levels of bone destruction and osteoporosis. Although these cells have the unique ability to restore bone, they share various characteristics with macrophages. As noted above, macrophages release nitric oxide in response to inflammatory cytokines and agents. A number of recent studies suggest that osteoclasts, like macrophages, synthesize nitric oxide (Kasten T P, Collin-Osdoby P, Patel N, Osdoby P, Krukowski M, Misko T P, Settle S L, Currie M G and Nickols G A (1994). Potentiation of osteoclast bone-resorption activity by inhibition of nitric oxide synthase. Proc Natl Acad Sci USA 91:3569-3573; Lowik C, Nibbering P H, van de Ruit M and Papapoulos S E. [1994] Inducible production of nitric oxide in osteoblast-like cells and in fetal mouse bone explants is associated with suppression of osteoclatic bone resorption. J Clin Invest 93:1465-1472); “In models of osteoporosis nitric oxide inhibition potentiated the loss of bone mineral density” (Kasten TP, Collin-Osdoby P, Patel N, Osdoby P, Krukowski M, Misko T P, Settle S L, Currie M G and Nickols G A (1994). Potentiation of osteoclast bone-resorption activity by inhibition of nitric oxide synthase. Proc Natl Acad Sci USA 91:3569-3573).

These studies show that inhibition of NOS activity in vitro and in vivo resulted in an apparent potentiation of osteoclast activity. Nitric oxide, on the other hand, strongly suppressed osteoclast activity and bone resorption. The exact relationship between nitric oxide, osteoclast activity and steroid hormones remains to be established. However, it seems likely that the steroid hormones may regulate nitric oxide synthesis in the osteoclasts and osteoblasts and this affects their activity. Collectively, these studies suggest that the down-regulation of nitric oxide synthesis in bones is associated with bone loss.

During early pregnancy, treatment with NOS inhibitors alone and in combination with low-dose antiprogestins resulted in a dose-dependent inhibition of implantation which indicate that nitric oxide is involved in implantation and nitric oxide deficiency may play a role in infertility and early pregnancy loss (K. Chwalisz, E. Winterhager and R. E. Garfield (1997) Nitric Oxide (NO) is involved in implantation: Interaction with progesterone. 44th Annual Meeting of Society for Gynecologic Investigation, Abstract #102,San Diego, Calif., The San Diego Marriott, Mar. 19-22, 1997). Generally, there is a high rate of spontaneous early abortion in fertile cycles in women. After natural conception, possibly as many as 50-60% of very early pregnancies are lost (Winston M L, Handyside A H [1993], New challenges in human in vitro fertilization. Science 260:932-935). On the other hand, human in vitro fertilization is surprisingly unsuccessful. This may be due to both conceptus abnormalities and dysynchrony between embryo and endometrium at the time of embryo transfer. The overall birth rate per IVF treatment cycle is approximately 14% in the USA (Medical Research International Society for Assisted Reproductive Technology [SART], The American Fertility Society [1992]. Fertil Steril 5:15), and 12.5% in UK (The Human Fertilization and Embryology Authority. Annual Report, London 1992).

Most early pregnancy losses may be due to abnormalities of the conceptus or the still inappropriate culture conditions, since the success of embryo transfer after IVF decreases as the time after insemination increases (Winston M L, Handyside A H [1993], New challenges in human in vitro fertilization. Science 260:932-935).

The effect of uterine environment on fertility rates after IVF may be equally important. It has been well established that the successful establishment of pregnancy after embryo transfer requires both a healthy blastocyst and a receptive uterus. Embryo transferred to an inadequately primed uterus are unlikely to implant. However, no effective methods to increase the implantation rates are available to date.

The most advanced stages of human implantation are characterized by the invasion of trophoblastic cells into the decidua and angiogenesis (Loke Y W, King A [1995] Human Implantation. Cell biology and immunology. Cambridge University Press). These stages are also dependent on progesterone, since progesterone antagonists also disrupt early pregnancy (Chwalisz K, Stöckemann K, Fuhrmann U, Fritzemeier K H, Einspanier A, Garfield R E [1995] Mechanism of action of antiprogestins in the pregnant uterus.In Henderson D, Philibert D, Roy A K, Teutsch G (eds) Steroid Receptors and Antihormones. Ann N.Y. Acad Sci 761:202-224). During early pregnancy, an adequate blood flow to the uterus is essential for embryo development. An impaired blood flow to the uterus can jeopardize the establishment of pregnancy (Edwards R G (1995) Clinical approaches to increasing uterine receptivity during human implantation. Hum Reprod 10, Suppl 3: 60-67). Patients with an impeded blood flow have been given aspirin to improve their blood flow (Goswamy R K, Williams G, Steptoe P C [1988], Decreased uterine perfusion-a cause of infertility. Hum. Reprod 3955-959). In conclusion, these data suggest that nitric oxide plays an important role in implantation and uterine perfusion during implantation and early pregnancy. Therefore, decreased nitric oxide production during early pregnancy may be associated with infertility and early pregnancy loss.

Primary or secondary nitric oxide deficiency may play a pivotal role in preeclampsia which represents after preterm birth a second major cause of perinatal mortality and morbidity. Treatment with NOS inhibitors during more advanced stages of pregnancy produce symptoms identical to preeclampsia in rats and guinea pigs (Chwalisz K and Garfield R E [1994], Role of progesterone during pregnancy: Models of parturition and preeclampsia. Z. Geburtsh. u. Perinat. 198:170-180). Preeclampsia is characterized by increased blood pressure and peripheral vascular resistance, fetal growth retardation, proteinuria and edema. In humans, histopathologic and clinical (fetal growth retardation, fetal death) evidence indicate that reduced placental perfusion is the earliest and most consistent change observed in preeclampsia (Roberts J M and Redman C W G. [1993], Pre-eclampsia: more than pregnancy-induced hypertension 341:1447-1451; Friedman E A [1988], Preeclampsia: a review of the role of prostaglandins. Obstet Gynecol 71:122-137). Preeclampsia is a common disease, which is generally identified in the latter half of pregnancy, affecting 5% -7% of pregnancies in developed countries. There are no effective methods for the prevention and treatment of preeclampsia and fetal growth retardation. The current therapy is restricted to bed rest (mild form), symptomatic medication with antihypertensive drugs and early delivery with attendant risks of operative delivery and iatrogenic prematurity. Aspirin, when given to inhibit prostaglandin synthesis in relatively low doses, is thought to predominantly suppress the platelet thromboxane A₂ production with little inhibition of the vascular prostacyclin production. Therefore, low-dose aspirin was proposed for the prevention of preeclampsia. The results of the recently published multicentric study are disappointing, and low-dose aspirin is currently not recommended for the prevention of preeclampsia (CLASP Collaborative Group: CLASP: a randomized trial of low-dose aspirin for the prevention and treatment of pre-eclampsia among 9364 pregnant women (1994). Lancet 343:619-629).

The L-arginine-nitric oxide system is present in the uterus and plays an important role in control of uterine contractility, pregnancy maintenance and the onset of labor. On the other hand, nitric oxide deficiency seems to be involved in preterm labor (Garfield R E and Yallampalli C. [1993] Control of myometrial contractility and labor. In: Basic Mechanisms Controlling Term and Preterm Birth. ed: K. Chwalisz, R E Garfield, Springer-Verlag, New York, pp. 1-29, Chwalisz K and Garfield R E. [1994], Antiprogestins in the Induction of labor. Ann New York Acad Scie 734:387-413; Buhimschi I, Yallampalli C, Dong Y-L and Garfield R E. [1995], Involvement of a nitric oxide-cyclic guanosine monophosphate pathway in control of human uterine contractility during pregnancy. Am J Obstet Gynecol 172:1577-1584, Sladek S M, Regenstrin A C, Lykins D. et al. [1993], Nitric oxide synthase activity in pregnant rabbit uterus decreases on the last day of pregnancy. Am J Obstet Gynecol 169:1285-1291). NOS inhibitors stimulates uterine contractility in vitro and in vivo, whereas L-arginine and nitric oxide inhibit uterine contractility in vitro and in vivo, suggesting that nitric oxide substitution can be used to stop preterm labor (Buhimschi I, Yallampalli C, Dong Y-L and Garfield RE [1995] Involvement of a nitric oxide-cyclic guanosine monophosphate pathway in control of human uterine contractility during pregnancy. Am J Obstet Gynecol 172:1577-1584; Garfield R E and Yallampalli C. [1993] Control of myometrial contractility and labor. In: Basic Mechanisms Controlling Term and Preterm Birth. ed:K. Chwalisz, R E Garfield, Springer-Verlag, New York, pp. 1-29,Sladek S M, Regenstrin A C, Lykins D. et al. [1993] Nitric oxide synthase activity in pregnant rabbit uterus decreases on the last day of pregnancy. Am J Obstet Gynecol 169:1285-1291; Natuzzi E S, Ursell P C, Harrison M. et al [1993], Nitric oxide synthase activity in the pregnant uterus decreases at parturition. Biochem Biophys ResCommun 194:108-114, Jennings R W, MacGillvray T E and Harrison M R. [1995], Nitric oxide inhibits preterm labor in the rhesus monkey. J Mat Fet Med 2:170-175).

Preterm labor, and subsequent preterm birth (i.e., birth before 37 completed weeks of gestation) are the major problems of perinatology overall. Preterm birth occurs with a frequency of about 10% in most European and North American countries and over 20% in less developed countries. With a world-wide birth rate of about 90 million babies per year, preterm labor is a major health issue, because it is the leading cause of infant mortality. It is estimated that approximately 13 million infants are born preterm world-wide each year (Berkovitz G S, Papiernik E. Epidemiology of preterm birth. Epidemiol Rev 1993; 15:414-443). In humans, premature birth is responsible for 75% of infant mortality and 50% of long-term neurological handicaps, including blindness, deafness, developmental delay, cerebral palsy, and chronic lung disease. The major causes of infant mortality are respiratory distress syndrome due to lung immaturity, and brain hemorrhage. Thus, any treatment which prolongs the length of pregnancy could have a profound effect on neonatal mortality and morbidity. The survival rate improves approximately by 2% per day from the 23rd to the 26th week of pregnancy (i.e. from 16% at 23 weeks to 57% at 26 weeks, reaching 80% at 28 weeks and over 90% after 30 weeks of gestation) (Haywood J L, Goldenberg R I, Bronstein J, Nelson K G, Waldemar A C Comparison of perceived and actual rates of survival and freedom from handicap, in premature infants. Am J Obstet Gynecol 1994; 171:432-439.).

Preterm labor has to be considered a syndrome of multifactorial origin. There is ample evidence that local or systemic infection, maternal and fetal stress, and low socio-economic status are associated with preterm labor and preterm birth. Presently, there is no effective treatment for preterm labor if a reduction in perinatal mortality is considered the chief criterion. A comprehensive review of tocolytic agents in the treatment of preterm labor, which analyzed 328 randomized, placebo-controlled studies (Higby K, Xenakis E M J, Pauerstein C J K Do tocolytic agents stop preterm labor?. A critical and comprehensive review of efficacy and safety. Am J Obstet Gynecol 1993; 168:1247-59), clearly demonstrates that current therapy is unsatisfactory. This analysis shows that: (a) magnesium sulfate is not superior to placebo, (b) β-adrenergic receptor agonists (betamimetics) effectively stop premature labor for only 24-48 hours, (c) the only tocolytic drugs that might be effective are the cyclooxygenase-inhibitors (indomethacin). Nevertheless, betamimetics, administered either intravenously or orally, and intravenous infusion of magnesium sulfate, two major methods of preterm birth treatment, are still widely used in obstetric clinics in combination with fetal lung maturation agents. Therefore, new tocolytic agents with improved efficacy and reduced side-effects are urgently needed.

In rats and guinea pigs, treatment with L-NAME inhibits cervical ripening and produces symptoms similar to cervical dystocia (Chwalisz K, Buhimschi I, Garfield R E (1996) Role of nitric oxide in obstetrics. Prenat Neonat Med 1,: 292-329). On the other hand, local application of nitric oxide induces cervical ripening (Chwalisz K, Shi Shao-Qing, Gerfield R E, Beier H M [1997] Cervical ripening after local application of nitric oxide. Hum Reprod 12: 101-109). In approximately 10-15% of term and near-term pregnancies, maternal and fetal conditions may require the induction of labor. The successful induction of labor with oxytocin or prostaglandins is largely dependent on the condition of the cervix, and if it is performed in the presence of an “unripe” cervix, the duration of labor is prolonged and there is a high failure rate. The two main approaches of cervical ripening currently applied in clinical practice involve the vaginal and endocervical administration of PGE₂. However, in about 20-30% of women, depending on the preparation and cervical status, repeated PGE₂ instillation is needed due to the low ripening effect. Some studies indicate an increased risk of uterine hyperstimulation and higher fetal heart rate abnormalities after the local application of PGE₂. Therefore, further refinements and improvements in cervical ripening are necessary, especially increased efficacy and a reduced risk of uterine hyperstimulation.

In women following menopause, there is an increase in cardiovascular diseases and in bone loss. Both effects might be related to a decline in sex steroids and a down regulation of NO. It is widely accepted that the beneficial effects of hormone replacement therapy (HRT) on cardiovascular disease in postmenopausal women results exclusively from the estrogen component of HRT. Indeed, large-scale epidemiological studies show that in postmenopausal women who receive estrogens both the cardiovascular and cerebrovascular mortality rates are reduced by 30-50% (Stamfer M J, Colditz G A. Estrogen replacement therapy and coronary heart disease: a quantitative assessment of the epidemiologic evidence. Prev Med 1991; 20:47-63). These effects cannot be fully explained by favorable changes in lipid profiles which seem to account for approximately 30-50% of the protective effects of estrogens (Barrett-Connor E, Bush T L. Estrogen and coronary heart disease in women J Am Med Assoc1991; 265:1861-1867). Recent studies indicate that the effects of estrogen on the cardiovascular system are at least in part mediated by nitric oxide. Estrogen exerts various effects on the blood perfusion of the reproductive tract, including regulating changes in uterine blood flow during both the pregnant and non-pregnant state. There is increasing evidence that estrogen has similar effects in other circulatory beds, including cerebrovascular and cardiovascular circulation (Collins P. Vascular effects of oestrogen. Maturitas 1996, 23:217-226). Numerous studies suggest that estrogens may stimulate vascular nitric oxide synthesis (Magness R R, Gant N F. Control of vascular reactivity in pregnancy: The basis for therapeutic approaches to prevent pregnancy-induced hypertension. Seminars in Perinatology 1994; 18 (2): 45-69; Gilligan D M, Badar D A, Guetta V, Quyyumi A A, Panza J A, Cannon R O Estradiol potentiation of endothelium-dependent vasodilation is dependent on nitric-oxide production. J Am Coll Cardiol 1994; Spec.Issue 378A). Since androgens can be converted to estrogens and can, on the other hand, exert a direct effects on nitric oxide synthesis, the increase in cardiovascular disease and osteoporosis may be due to the reduction in nitric oxide as a result of androgen deprivation during aging. It is also known that nitric oxide is involved in penile erection and that androgens up-regulates nitric oxide production in the penis. Male impotence as a result of androgen deprivation as a frequent problem of aging men. In conclusion, a generalized NO deficiency may be considered as a hallmark of climacterium in both women and men.

The pathological conditions due to nitric oxide deficiency can be treated with agents which either release nitric oxide such as nitric oxide donors or raise endogenous blood and/or tissue levels of nitric oxide. The pathway of nitric oxide production via the biochemical transformation of L-arginine by NOS is well established. However, relatively little is known about the pathway(s) by which cells synthesize or metabolize L-arginine. Cultured endothelial cells can generate L-arginine from an intracellular source which can be linked to the release of EDRF. These cells can also convert (recycle) L-citrulline to L-arginine in an arginine-citrulline cycle through the intermediate formation of arginosuccinate which involves the urea cycle enzymes arginosuccinate synthase (conversion of citrulline to arginosuccinate) and arginosuccinase (conversion of arginosuccinate to arginine) (Hecker et al., 1990; Bredt D S, Schmidt H H H W, [1996] The metabolism of L-arginine and its significance for the biosynthesis of endothelium-derived relaxing factor: Cultured endothelial cells recycle L-citrulline to L-arginine. Proc Natl Acad Sci. USA 87:8612-8616). These two enzymes which allow the recycling of citrulline to arginine are present in various NOS-containing cells, including endothelial cells (Hecker et al., 1990) and some neurons (Arnt-Ramos L R, O'Brien W E, Vincent S R (1992).Immunohistochemical localization of arginosuccinate synthase in the rat brain in relation to nitric oxide-containing neurons. Neuroscience 51:773-789). While these in vitro studies show that citrulline can be re-cycled to arginine, their did not lead to any conclusions concerning any therapeutic use of citrulline.

EP 0441 119 A2 discloses the use of L-arginine in the treatment of hypertension and other vascular disorders. It suggests that the mechanism by which L-arginine is effective for this purpose is because it may be the physiological precursor of the most powerful endothelial-derived releasing factor, nitric oxide. U.S. Pat. No. 5,508,045 discloses the use of nitric oxide substrates and donors for control and management of labor during pregnancy. U.S. Pat. No. 5,595,970 discloses the treatment of climacteric disorders with nitric oxide synthase substrates and/or with nitric oxide donors. See also U.S. application Ser. Nos. 08/466,538, 08/152,496, 08/466,689, 08/310,950, 08/437,462, 08/588,586, 08/812,912, 08/812,910, 08/812,910 and 08/092,426. However, there are no disclosures demonstrating that nitric oxide deficiency symptoms can be attenuated by raising the endogenous nitric oxide levels with citrulline treatment. In addition, the therapeutic value of citrulline in various nitric oxide deficiency states has not been recognized to date.

### Objects of the Invention

The present invention provides a novel method for control, treatment, management and prevention of various conditions related to nitric oxide deficiency. The method comprises to a mammal citrulline or a citrulline analogue alone or in combination with other enhancing and modulating agent.

It is an object of the invention to provide a method for the prevention and treatment of hypertension and other high vascular resistance disorders, including primary or secondary vasospasm, angina pectoris, cerebral ischemia, restenosis, with citrulline or a citrulline analogue.

It is an object of the invention to provide a method for the prevention and treatment of the cardiovascular disease in women and men with citrulline or a citrulline analogue.

It is another object to provide such a method in which an estrogenic, partial estrogenic, or androgenic agent is used in combination with citrulline or a citrulline analogue for the prevention and treatment of the cardiovascular disease.

It is another object to provide a method for the treatment and/or prevention of preeclampsia, HELLP syndrome, and intrauterine growth retardation with citrulline or a citrulline analogue.

It is another object to provide such a method in which a progestational agent and or a cyclooxygenase inhibitor is used in combination with citrulline or a citrulline analogue for the treatment and/or prevention of preeclampsia, HELLP syndrome, and intrauterine growth retardation.

It is a further object to provide a method for the treatment of uterine contractility disorders, including preterm labor and dysmenorrhea with citrulline or a citrulline analogue.

It is another object of the invention to provide a method for treatment and prevention of infertility or early pregnancy loss with citrulline or a citrulline analogue in early pregnant mammals.

It is a further object of the invention to provide a method for improvement of implantation and treatment and prevention of infertility or early pregnancy loss with citrulline or a citrulline analogue in which progesterone or a progestagenic agent in combination with citrulline or a citrulline analogue is used.

It is another object of this invention in which a progestational agent and an estrogenic agent are used in combination with citrulline or a citrulline analogue for the for improvement of implantation and treatment and prevention of infertility or early pregnancy loss in female mammals.

It is another object to provide a method for the prevention and treatment of male and female climacterium.

It is a further object to provide a method for the treatment and prevention of osteoporosis with citrulline or a citrulline analogue.

It is another object to provide such a method in which an estrogenic or androgenic agent is used in combination with citrulline or a citrulline analogue for the prevention and treatment of osteoporosis.

It is a further object to provide a method for hormone replacement therapy (HRT) in peri- and post-menopause using a estrogenic, or partial estrogenic agent in combination with citrulline or a citrulline analogue.

It is another object to provide a method for HRT in the peri- and post-menopause using a combination of an estrogenic agent, with or without an additional progestational agent, with citrulline or a citrulline analogue.

It is a further object to provide a method for the treatment of male erectile impotence with citrulline or a citrulline analogue.

It is a further object to provide a method for cervical ripening by administering locally citrulline or a citrulline analogue.

It is another object to provide a method to manage and regulate vascular conductance, blood flow, and blood pressure with citrulline or a citrulline analogue.

It is a further object to provide a method for the treatment of gastrointestinal tract disorders, including altered motility, pyloric stenosis, and diabetes mellitus.

It is a further object to provide a method for the treatment of respiratory system diseases, including asthma, neonatal respiratory distress syndrome, pulmonary hypertension, and adult respiratory distress syndrome.

It is a further object to provide a method for the treatment of inflammatory diseases, including acute inflammation, resistance to infection, SLE-lupus, anaphylactic reaction, allograft rejection.

It is a further object to provide a method for the treatment or management central nervous system conditions associated with nitric oxide deficiency, including Alzheimer's disease, stroke, growth hormone disorders, and behavior problems.

It is a further object to provide a method for the treatment of dermatological conditions such as atopic eczema, topical hair loss, and burn injury.

A further object is the provision of pharmaceutical compositions useful in practicing the methods of this invention.

Other objects will be apparent to those skilled in the art to which this invention pertains.

### SUMMARY OF THE INVENTION

In a method aspect, this invention relates to a method for control, management, treatment and prevention of disorders and diseases related to nitric oxide deficiency or those disorders or diseases which can be improved by enhancing endogenous nitric oxide synthesis.

Yet another aspect of the current invention is a method for control, management, treatment and prevention of conditions related to nitric oxide deficiency such as hypertension, cardiovascular disease, osteoporosis, diabetes mellitus, preeclampsia HELLP, syndrome and fetal growth retardation; uterine contractility disorders such as preterm labor and dysmenorrhea, cervical dystocia, infertility and early pregnancy loss; male impotence; urinary incontinence; intestinal tract disorders (e.g. altered motility and pyloric stenosis), respiratory system diseases (e.g. asthma, neonatal respiratory distress syndrome, pulmonary hypertension, and adult respiratory distress syndrome); inflammatory diseases (e.g. acute inflammation, resistance to infection, SLE-lupus, anaphylactic reaction, allograft rejection); Alzheimer's disease, stroke, growth hormone disorders, and behavior changes; dermatological conditions such as atopic eczema, topical hair loss, and burn injury; by administering citrulline or a citrulline analogue.

Still another aspect of the current invention is a method for control, management, treatment and prevention of conditions related to nitric oxide deficiency in combination with other enhancing or modulating agents.

Still yet another aspect of the current invention is a method for the prevention and treatment of female and male climacterium (climacteric symptoms) by administering citrulline or a citrulline analogue alone or in combination with an estrogenic, estrogenic and progestagenic, and androgenic agent.

In a product aspect, this invention relates to a pharmaceutical composition comprising citrulline or at least one of the a citrulline analogue, which produce, control or alter nitric oxide availability alone or in further combination with one or more of a estrogen and/or progestin, and/or androgen, and/or other enhancing or modulating agents which potentiate nitric oxide action.

### DETAILED DISCLOSURE

The methods of this invention control, manage, treat or prevent conditions due to nitric oxide deficiency in a mammal, preferably a human, who is manifesting the symptoms thereof or who is a high risk candidate for doing so.

Because these abnormal conditions are produced by or aggravated by subnormal nitric oxide synthesis, both citrulline (200 mg-10 g p.o.) and citrulline analogues (at bioequivalent doses) which serve as precursors of the natural nitric oxide substrate, L-arginine are useful for ameliorating the symptoms thereof and, in one aspect of the method of this invention, a combination of citrulline or a citrulline analogues with other enhancing and modulating agents is employed.

The term citrulline and citrulline analogues refers to compounds of formula I, their D,L racemic mixture, their L-isomers and salts thereof like for instance but not limited to salts of inorganic or organic acids like oxalic acid, lactic acid, citric acid, fumaric acid, acetic acid, phosphonic acid, HCl, HBr, sulfuric acid, p-toluol-sulfonic acid. Also suitable are salts of bases such as sodium-, potassium-, or calcium hydroxide, of ammonia, or of amines like ethanolamine, diethanolamine, tri-ethanolamine, N-methylglucamin, tris-(hydroxymethyl)-methylamine or bis-cyclohexylamine just to name a few.

Citrulline and citrulline analogues useful in the present invention include, e.g., compounds such as:

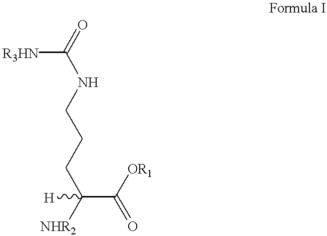

wherein

R₁ has the meaning of hydrogen, alkyl, alkenyl, aryl, phenacyl, omega-hydroxyalkyl or omega-methoxyalkyl,

R₂ and R₃ may be selected independently from hydrogen, alkyl, aryl, acetyl, benzoyl, tert. -butoxycarbonyl, wherein

alkyl means a branched or linear chain with 1 to 10 carbons;

alkenyl means a branched or linear chain with 1 to 10 carbons containing up to 3 double bonds;

aryl means a phenyl or naphthyl moiety, optionally substituted once or twice with methyl, nitro, amino or chlorine,

benzoyl may be optionally substituted once or twice with methyl, nitro, amino or chlorine,

phenacyl means a group of formula —(CH₂)ₙ(C═O)aryl, and

n can be 1 to 3.

The compounds are commercially available or can be synthesized from citrulline or from ornithine with known methods. The salts can be bought or are easily available by stirring the amino acid with another acid or base in solvents, such as, e.g., ethanol or dioxane or dialkylether or tetrahydrofuran. The following compounds may be taken as examples:

D,L-citrulline,

L-citrulline,

L-citrulline, monoacetate

L-citrulline, hydrochloride

L-citrulline methylester,

L-citrulline ethylester,

L-citrulline-n-hexylester,

L-citrulline (benzoylmethyl)ester, alpha-N-benzoyl-L-citrulline methylester,

N-Boc-L-citrulline,

N1-2,4-dinitrophenyl-D,L-citrulline.

The present invention provides a novel method for control, management, treatment and prevention of different nitric oxide-dependent disorders and diseases such as hypertension, cardiovascular disease (e.g. atherosclerosis, restenosis), osteoporosis, preeclampsia, HELLP syndrome and fetal growth retardation, uterine contractility disorders such as preterm labor and dysmenorrhea, cervical dystocia, male impotence, urinary incontinence, etc. In addition, this invention provides a novel method for treatment of infertility by improving implantation rates or controlling ovulation.

In case of hypertension and cardiovascular disease the method comprises administering citrulline or a citrulline analogue to a female or male mammal experiencing these conditions alone or in combination with agents enhancing or modulating endogenous NO production, and/or in combination with nitric oxide donors. Such agents include, but are not limited to such as for example S-nitroso-N-acetyl-penicillamine, nitroglycerin, diethyloamino nitric oxide, and other analogues thereof, and other substrates of NO such as L-arginine.

In case of the treatment of uterine contractility disorders during pregnancy (preterm birth) and in the non-pregnant state (dysmenorrhea and dysfunctional bleeding), the present method comprises administering citrulline or a citrulline analogue to a female mammal alone or in combination nitric oxide donors, e.g., sodium nitro-prusside, nitroglycerin, glyceryl trinitrate, SIN-1, isosobid mononitrate and isosorbid dinitrate, for ameliorating the symptoms thereof. A synergistic effect may also be achieved administering citrulline or a citrulline analogue in combination with one or more of a prostaglandin inhibitor, a progestin, an oxytocin antagonist or a β-agonist.

Thus, the method aspect of this invention and the pharmaceutical composition aspect of this invention employs (a) either or both of a nitric oxide donor and a nitric oxide substrate and, optionally (b) one or more of a prostaglandin inhibitor (e.g. aspirin, indomethacin, or ibutrophen), a progestin (progesterone, norgestrel, medaxyprogesterone, etc.), an oxytocin antagonist (e.g. atosiban, etc.) or a β-agonist (e.g. salbutinol, terbutaline, etc.).

The present invention additionally provides a method for the improvement of the implantation and birth rates after IVF and to prevent early pregnancy loss in a pregnant female who is manifesting the symptoms thereof. Because the low implantation and birth rates and early pregnancy loss are produced by or aggravated by inadequate uterine blood supply to the conceptus due to subnormal nitric oxide synthesis, citrulline or a citrulline analogue alone or in further combination with both nitric oxide synthase substrates, e.g., L-arginine and nitric oxide donors, e.g., sodium nitroprusside, nitroglycerin, glyceryl trinitrate, SIN-1, isosorbid mononitrate isosorbid dinitrate and diethylenetriamine/NO (DETA/NO), are useful for ameliorating the symptoms thereof and, in one respect of this method of this invention, a combination of both are employed. An additive additional effect is achieved when a progestagenic and/or and estrogenic agent is administered concurrently administering citrulline or a citrulline analogue for the treatment of infertility and implantation problems. In the case of a female mammal, a progestagenic agent can be administered concurrently with or in lieu of an estrogen. Thus, the method aspect of this invention and the pharmaceutical composition aspect of this invention employs either citrulline or a citrulline analogue and, optionally one or more of an estrogen (e.g., Progynova^{R}, Schering) or a progestin (e.g. progesterone or hydroxyprogesterone caproate [Proluton_{R} Depot], etc.).

The present invention additionally provides a method for the control and management of cervical ripening and for the treatment of cervical dystocia by administering locally (intracervically, vaginally) citrulline or a citrulline analogue to a female mammal alone or in combination nitric oxide donors, e.g., sodium nitroprusside, nitroglycerin, glyceryl trinitrate, SIN-1, isosobid mononitrate and isosorbid dinitrate. A synergistic effect may be achieved administering citrulline or a citrulline analogue is used in combinations with one or more of a prostaglandin (e.g. PGE₂ gemeprost), an interleukin (e.g. IL-8), or an antiprogestin (e.g. mifepristone, ZK 137 316, ZK 230 211, ORG 33628, etc.)

Further, the present invention provides a method to treat climacterium (climacteric symptoms) in a menopausal/postmenopausal female or in a male human, who is manifesting the symptoms thereof or who is a high risk candidate for doing so. e.g. based on rate of bone loss rate.

Because abnormal conditions of the menopause/male climacterium are produced by or aggravated by subnormal nitric oxide synthesis, both citrulline and citrulline analogues which serve as precursors of the natural nitric oxide substrate, L-arginine, are useful for ameliorating the symptoms thereof and, in one aspect of the method of this invention, a combination of citrulline or a citrulline analogue with the steroid hormones and other compounds used for hormone replacement therapy is employed. In female mammals, an additional effect is achieved when a estrogen, estrogen partial antagonist, and/or progestational agent is administered concurrently with citrulline or a citrulline analogue. In the case of a male mammal, an androgen or a compound increasing endogenous androgen levels, for example an aromatase inhibitor, can be administered concurrently with citrulline or a citrulline analogue.

Thus, the method aspect of this invention and the pharmaceutical composition aspect of this invention employs either or both of citrulline or citrulline analogue and a nitric oxide donor and, optionally, one or more of a estrogen (e.g. Progynova®, Schering), partial estrogen against (Raloxifen, tamoxifen, centchromane, clomiphene citrate, droloxifene or other related compounds), and a progestin (e.g., progesterone or norgestrel).

Examples of combinations of active agents which can be administered concurrently with citrulline or a citrulline analogue and/or nitric oxide donor are estrogens and progestins (see below).

Examples of dosage ranges of typical nitric oxide-substrates and nitric oxide-donors (per os) are.

| active compound | dose per day |
|---|---|
| L-Arginine | 500 mg-10 g/day p.o. |
| Sodium nitroprusside | 500-2000 μg/kg/day |
| Nitroglycerin | 0.5-10 mg/day |
| Isosorbid mononitrate | 10-100 mg/day |
| Isosorbid dinitrate | 10-100 mg/day |

The following are typical oral dosage ranges active agents of the estrogen and progestin with the nitric oxide substrate or donor:

Estrogens: a daily dose bioequivalent to about 1 to 2 mg per day, e.g., Premarin®, Wyeth-Ayerst, 0.625 mg/day, estradiol valerate, 50 μg/day transdermally, vaginal estradiol creams, 1.25 mg/day and vaginal estradiol rings, 0.2 mg/day.

Partial estrogens: Raloxifene (Eli Lilly) (daily dose 0.1-600 mg/day orally, preferably 10-100 mg), tamoxifene at a daily dose of 1-200 mg/day orally, levormeloxifene (Novo-Nordisk) at a daily dose of 1-200 mg/day orally, clomiphene citrate at a daily dose of 1-200 mg, zuclomiphene citrate at a daily dose of 1-200 mg, droloxifene (3-hydroxy tamoxifene), CP 336 156 (Pfizer/Klinge), Idoxifene (Smith Kline) and RU 39 411 at equivalent doses.

Progestins: A daily dose bioequivalent to 50-300 mg of progesterone/day, e.g., an indictable suspension of medroxyprogesterone acetate to provide a weekly dose of thereof of 100-1000 mg or tablets or dragees providing an oral dose thereof of 5-10 mg/day, an injectable solution of hydroxyprogesterone caproate which provides a weekly dose of 250-500 mg; tablets, capsules or dragees of northindrone acetate which provide a daily dose of 5-20 mg.

Androgens: Testosterone (Testoderm, Alza Pharmaceuticals; testosterone transdermal system) at a daily dose 1-10 mg (preferable 4 mg/day). Testosterone propionate (Testoviron-Depot-250; Schering) at a dose of 10-250 mg i.m. injections every 2-4 weeks or 10-100 mg i.m. injections 2-3 times per week. Testosterone enanthate (Delatestryl) 100-250 mg every 2 weeks i.m. Testosterone cypionate (Depo-Testosterone, Upjohn) 100-250 mg every 1-3 weeks i.m. Testosterone undecanoate (Andriol, Organon) 20-200 mg orally. Mesterolon (Proviron 25; Schering) 25-200 mg/day orally. Methyltestosterone (1-100 mg orally).

Other examples of estrogens and progestins are listed below: Oral “natural” estrogens used in hormone replacement therapy currently available in the UK.

| Product | Composition | Dose (mg per day) |
|---|---|---|
| Climaval (Sandoz) | Estradiol valerate | 1 or 2 |
| Progynova (Schering) | Estradiol valerate | 1 or 2 |
| Harmogen (Abbott) | Piperazine estrone sulfate | 1.5 or 2.5 |
| Hormonin (Shire) | Estradiol+ | 0.6 |
| | Estrone+ | |
| | Estriol | |
| Premarin (Wyeth-Ayerst) | Conjugated equine | 0.625 or 1.25 or 2.5 |
| | estrogens | |

Commercially available combination calendar packs for hormone replacement therapy:

| Product | Generic composition |
|---|---|
| Premarin | Conjugate equine estrogen (o.625 or 1.25 or 2.5 |
| | mg/day), oral |
| Estrace/Zumenon | Estradiol valerate 1 or 2 mg/day, oral |
| Prempack C | Conjugated equine oestrogen (o.625, 1.25 mg plus |
| | levonorgestrel o.15 mg) |
| Estraderm | Estradiol 0.025, 0.1 mg/day, transdermal |
| Systen/Evorel | Estradiol 0.05 mg/day transdermal |
| Premarin vaginal | Conjudated equine oestrogen 0.625 mg/day, vaginal |
| Trisequens | Estradiol 2 mg per day (10 days); Norethisetrone |
| | acetrate 1 mg per day plus 23 mg estradiol (12 days); |
| | Estradiol 1 mg per day (6 days); oral |
| Trisequens forte | Estrodiol 4 mg per day (10 days); Norethiserone |
| | acetate 1 mg per day plus 4 mg estradiol (12 days); |
| | Estradiol 1 mg per day (6 days); oral |
| Nuvelle | Estradiol valerate 2 mg/day plus levonorgestrel 0.75 |
| | mg/day, oral |
| Cyclo-Progynova | Estradiol valerate 2 mg per day (11 days); Estradiol |
| | valerate 2 mg per day plus Levonoregestrel 0.5 mg per |
| | day (10 days), oral |

Daily doses of progestogens taken for 12 days per month in patients receiving oral or transdermal estrogens.

Norethisterone 0.7-2.5 mg per day

Medroxyprogesterone acetate 10 mg per day

Norgestrel 150 mg per day

Dydrogesterone 10-20 mg per day

Examples of cardiovascular agents:

| Pharmacological group/generic name | dose |
|---|---|
| adrenergic blockers: | |
| guanethidine (Ismelin, Ciba-Geigy) | 10-25 mg once-a-day |
| alpha blockers: | |
| labelatol HC2 | 100 mg 2 ×/day |
| adrenergic inhibitors | |
| Methyldopa (Aldomet, Merck) | 250 mg 2-3 ×/day |
| angiotensin converting enzyme inhibitors | |
| (ACE-inhibitors) | |
| Ovinapril HCl (Accupril, Parke-Davis) | 10 mg/day |
| Ramipiril (Altace, Hoechst-Roussel) | 2.5 mg/day |
| beta blockers: | |
| Propranolol (Inderal; Wyeth, Ayrst) | 40 mg 2 ×/day |
| calcium channel blockers: | |
| Nicardipine (Cardene, Syntex) | 20 mg 3 ×/day |
| duretics: | |
| chloratiazide (Diuril, Merck) | 0.5-1 g/day |
| anthiarrhytmics: | |
| Disopyramide (Norpace, Searle) | 150 mg every 6 hours |
| direct vasodilators: | |
| Hyralazine (Apresoline, Ciba Geigy) | 10-25 mg 4 ×/day |

The pharmacologically active agents employed in this invention can be administered in admixture with conventional excipients, i.e., pharmaceutically acceptable liquid, semi-liquid or solid organic or inorganic carriers suitable, e.g., for parental or enteral application and which do not deleteriously react with the active compound in admixture therewith. Suitable pharmaceutically acceptable carriers include but are not limited to water, salt solutions, alcohols, vegetable oils, polyethylene glycols, gelatin, lactose, amylose, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc.

Pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds.

For parenteral application, particularly suitable are solutions, preferably oily or aqueous solutions, as well as intravaginal or intracervical gels, suspensions, emulsions, or implants, including suppositories and transdermal patches. Ampoules are convenient unit dosages. In a preferred aspect, the composition of this invention is adapted for ingestion.

For internal application, particularly suitable are unit dosage forms, e.g., tablets, dragees or capsules having talc and/or a carbohydrate carrier or binder or the like, the carrier preferably being lactose and/or corn starch and/or potato starch; particulate solids, e.g., granules; and liquids and semiliquids, e.g., syrups and elixirs or the like, wherein a sweetened vehicle is employed. Sustained release compositions can be formulated including those wherein the active compound is protected with differentially degradable coatings, e.g., by microencapsulation, multiple coatings, etc.

Suitable for oral administration are, inter alia, tablets, dragees, capsules, pills, granules, suspensions and solutions. Each unit dose, e.g., each tablespoon of liquid or each tablet, or dragee contains, for example, 5-5000 mg of each active agent.

Solutions for parenteral administration contain, for example, 0.01-1% of each active agent in an aqueous or alcoholic solution.

The nitric oxide substrate and/or donor can be administered as an admixture with an estrogen and/or progestational agent and any other optional active agent or as a separate unit dosage form, either simultaneously therewith or at different times during the day from each other.

The combination of active agents is preferably administered at least once daily (unless administered in a dosage form which delivers the active agents continuously) and more preferably several times daily, e.g., in 2 to 6 divided doses. The typical dose is about 0.5 to 1000 mg of each active agent, although some less active agents, e.g., L-Arginine, require much higher oral dosages, e.g., 500 to 10,000 mg, and others, e.g., sodium nitroprusside, require lower doses, e.g., 500-2000 μg/kg/day. Doses for nitroglycerin typically are orally 2.5 mg 2×daily; sublingually, 0.8 mg 1-4×daily; and transdermally, 0.2-0.4 mg/hr. Since the LD₅₀ dosages of most of these active agents is known in the prior art, a lower dosage regimen can be initiated and the dosage increased until a positive effect is achieved or a higher dosage regimen can initially be employed, e.g., in a crisis situation, and the dosages regulated downward as relief from the symptoms is achieved. Combinations of agents can be employed either continuously or sequentially.

### BRIEF DESCRIPTION OF DRAWINGS

Various other features and attendant advantages of the present invention will be more fully appreciated as the same becomes better understood when considered in conjunction with the accompanying drawings, in which like reference characters designate the same or similar parts throughout the several views, and wherein:

FIG. 1: The effect of a single administration of citrulline on the systolic blood pressure in normal and L-NAME-treated female rats. Intact female rats chronically instrumented with telemetric transducers allowing continuous monitoring of blood pressure were subcutaneusly implanted with osmotic mini-pumps releasing 50 mg/day L-NAME or 0.9% saline (controls). 24 hours after the pump insertion, the animals received 70 mg/rat citrulline orally per gavage. The systolic blood pressure was monitored for an additional 24 hours following citrulline treatment. N=5/group.

FIG. 2: The effect of multiple administration of citrulline on the systolic blood pressure in normal and L-NAME-treated female rats. Intact female rats chronically instrumented with telemetric transducers allowing continuous monitoring of blood pressure were subcutaneously implanted with osmotic mini-pumps releasing 50 mg/day L-NAME or vehicle (controls). 24 and 48 hours after the pump insertion, the animals received 70 and 140 mg/rat citrulline orally per gavage. The systolic blood pressure was monitored for an additional 24 hours following the second citrulline treatment. N=5/group

FIG. 3: The effect of a single administration of citrulline on the heart rate in normal and L-NAME-treated female rats. Intact female rats chronically instrumented with telemetric transducers allowing continuous monitoring of heart rate were subcutaneously implanted with osmotic mini-pumps releasing 50 mg/day L-NAME or 0.9% saline (controls). 24 hours after the pump insertion, the animals received 70 mg/rat citrulline orally per gavage. The heart rate was monitored for an additional 24 hours following citrulline treatment. N=5/group

### Discussion of the Drawings

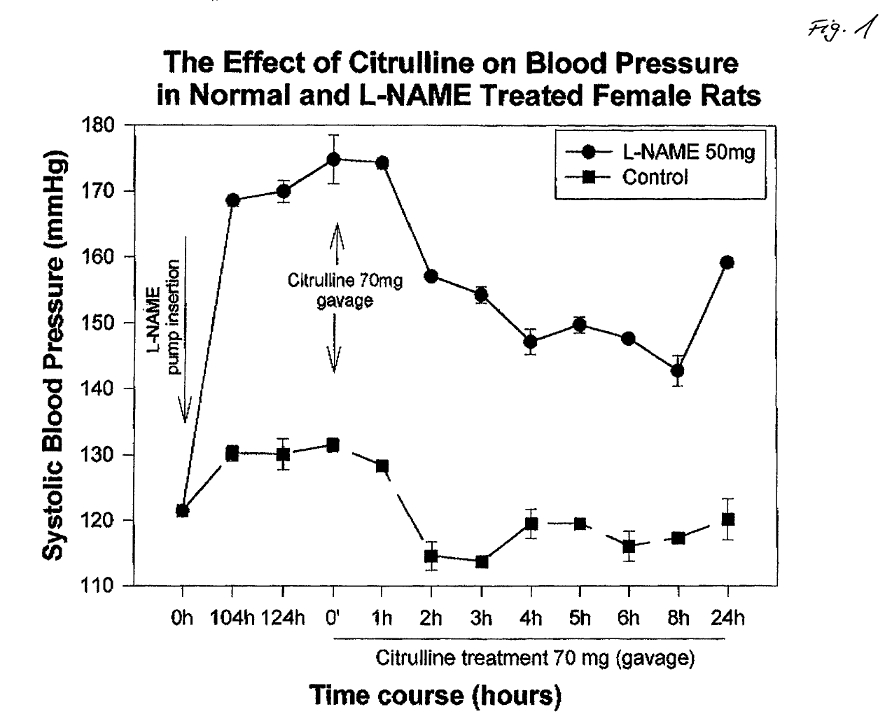

In the experiment which results are shown by the graph of FIG. 1, the animals were treated with citrulline 24 hours after the start of L-NAME infusion, i.e. when a substantial hypertension was produced by the NOS inhibition. Following a single oral citrulline (70 mg) administration, there was a rapid and significant (p<0.05) decrease in systolic blood pressure with a plateau reaching between 4 and 8 hours after treatment, with subsequent rise 24 hours after treatment. Citrulline also reduced blood pressure in control animals which were infused with the vehicle. However, the systolic BP started to raise 4 hours after citrulline treatment.

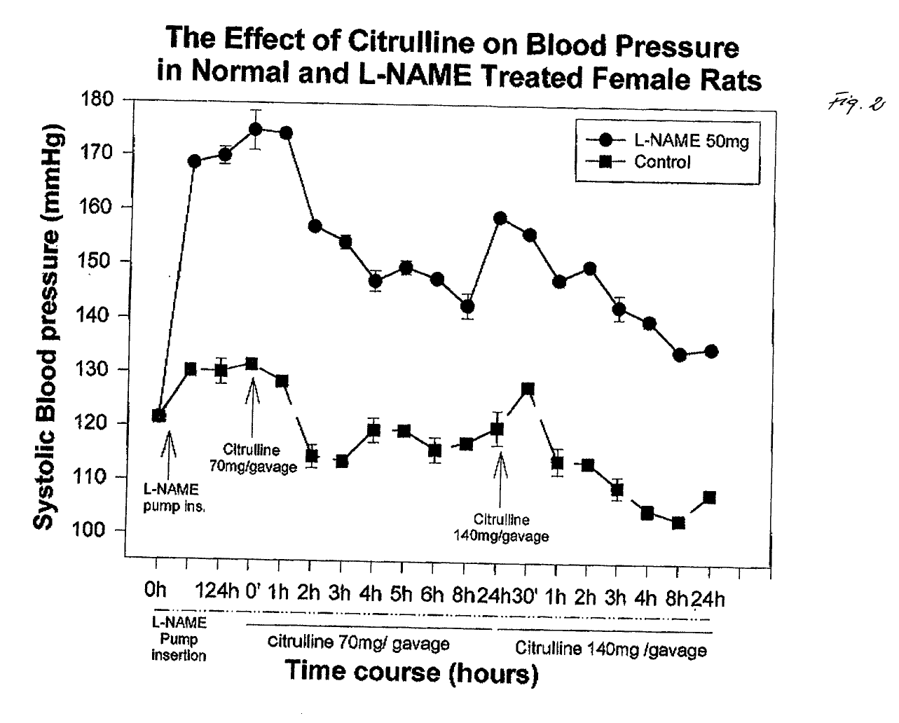

In the experiment which results are presented in FIG. 2, the animals were treated twice with citrulline, i.e. 24 hours (70 mg/rat) and 48 after the start of L-NAME infusion. Similarly to FIG. 1, the first administration of citrulline was performed when the animals became hypertensive following L-NAME infusion. In both L-NAME- and vehicle-treated animals multiple administration of citrulline substantially decreased blood pressure by approximately 30-40%, the second dose of 140 mg being slightly more effective.

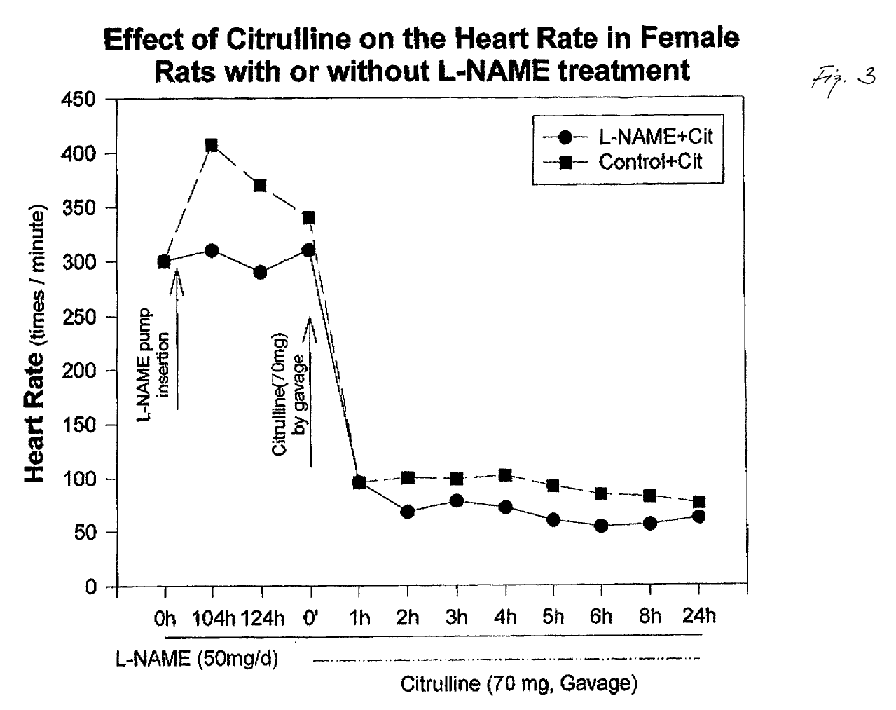

In the experiment which results are shown by the graph of FIG. 3, the animals were treated with citrulline 24 hours after the start of L-NAME infusion, i.e., when a substantial hypertension was produced by the NOS inhibition. At this time there was also a pronounced increase in heart rate in the L-NAME-treated rats (to 400-450 times/minute). Following a single oral citrulline (70 mg) administration, there was a rapid and very pronounced (p<0.05) decrease in heart rate to approximately 100 times/minute reaching a plateau already 1 hour after treatment. Citrulline also reduced heart rate in control animals which were infused with the vehicle. The effects of citrulline sustained for 24 hours in both treatment and control groups of citrulline treatment.

No side-effects of citrulline administration could be observed in any experiment. It can be concluded from these studies that citrulline, administered orally, partially attenuated hypertension and an increase in heart rate induced by NOS inhibition with L-NAME.

Therefore, the method of treatment employed in this invention can also be employed for the treatment of hypertension (in both females and males), and a variety of other disorders related to nitric oxide deficiency such as cardiovascular disease, thrombotic disorders and hemorrhage, preeclampsia, preterm labor and dysmenorrhea, as an adjuvant in hormone replacement therapy (HRT in both females and males) etc., following the dosage regime described herein.

Without further elaboration, it is believed that one skilled in the art can, using the preceding description, utilize the present invention to its fullest extent. The preferred specific embodiments are, therefore, to be construed as merely illustrative, and not limitative of the disclosure in any way whatsoever.

The entire disclosure of all applications, patents and publications, cited above and below are hereby incorporated by reference.

### EXAMPLES

### Example 1

Treatment of Hypertension

To a human male (ca 45 years; 70-80 kg) displaying hypertension, administer 0.5 to 20 g of citrulline per os daily in three divided doses until the symptoms are ameliorated. Thereafter administer 0.5 to 5 g of citrulline daily.

### Example 2

Treatment of Cardiovascular Disease

To a human male (ca 45 years; 70-80 kg) displaying symptoms of cardiovascular disease (e.g. chest pain), administer 0.5 to 20 g of citrulline per os daily in three divided doses until the symptoms are ameliorated. Thereafter administer 0.5 to 5 g of citrulline daily.

### Example 3

Treatment of Cardiovascular Disease with Citrulline in Combination with a Nitric Oxide Donor

To a human male (ca 45 years; 70-80 kg) displaying symptoms of cardiovascular disease (e.g. chest pain), administer 0.5to 5 g of citrulline in three divided doses in combination with nitroglycerin (1-10 mg transdermal patch) until the symptoms are ameliorated.

### Example 4

Prevention of the Cardiovascular Disease with Citrulline in Combination with an Estogenic Agent

To a nonpregnant human female (ca 45 years; 50-80 kg) displaying clinical signs of early cardiovascular disease or at risk to develop it (e.g., hypercholesterolemia, heavy smoking, etc.), administer citrulline 0.5 to 20 g per os daily in three divided doses in combination with estrogen (e.g. estradiol valerate) 1-2 mg daily.

### Example 5

Treatment of Preeclampsia with Citurlline

To a pregnant human female displaying clinical signs preeclampsia (hypertension, proteinuria, fetal growth restriction, edema), administer citrulline 0.5 to 20 g per os daily in three divided in combination with estrogen (e.g., estradiol valerate) 1-2 mg daily.

### Example 6

Prevention of Preeclampsia with Citrulline

To a pregnant human female in the second trimester of pregnancy at risk to develop preeclampsia (e.g., increased resistance of the uterine arteries in Doppler ultrasound) administer citrulline 0.5 to 20 g per os daily until delivery.

### Example 7

Prevention of Preeclampsia with Citrulline in Combination with a Nitric Oxide Donor

To a pregnant human female in the second trimester of pregnancy at risk to develop preeclampsia (e.g., increased resistance of the uterine arteries in Doppler ultrasound), administer citrulline 0.5 to 20 g per os daily in combination with nitroglycerin 1-10 mg transdermally until delivery.

### Example 8

Treatment of Preterm Labor with Citrulline

To a pregnant human female displaying clinical signs of labor (regular uterine contractions), administer citrulline 0.5 to 20 g per os daily alone or in combination with a tocolytic agent (e.g., ritodrine, nitroglycerin, magnesium sulfate).

### Example 9

Treatment of Preterm Labor with Citrulline

To a pregnant human female displaying clinical signs of labor (regular uterine contractions) administer citrulline 0.5 to 20 g per os daily alone or in combination with a tocolytic agent (e.g. ritodrine, nitroglycerin, magnesium sulfate).

### Example 10

Treatment of Preterm Labor with Citrulline in Combination with Progesterone

To a pregnant human female displaying clinical signs of labor (regular uterine contractions) administer citrulline 0.5 to 20 g per os daily alone or in combination with progesterone (e.g., Proluton Depot (Schering) 250-1000 mg/week i.m.).

### Example 11

Treatment of Dysmenorrhea with Citrulline

To a non-pregnant human female suffering from dysmenorrhea administer citrulline 0.5 to 20 per os daily.

### Example 12

Improvement of Implantation Rates After in Vitro Fertilization with with Citrulline.

To a pregnant human female (50-90 kg) undergoing IVF, administer citrulline 0.5 to 20 g per os daily in three divided doses for the first 2-6 weeks of pregnancy or longer.

### Example 13

Treatment of Infertility with Citrulline.

To a infertile human female (50-90 kg), administer citrulline 0.5 to 20 g per os daily in three divided doses

### Example 14

Improvement of Implantation Rates After in Vitro Fertilization with Citrulline in Combination with Progesterone.

To a pregnant human female (50-90 kg) undergoing IVF, administer citrulline in combination with progesterone (e.g. Proluton®Depot (Schering) 250-1000 mg/week i.m.) for the first 2-6 weeks of pregnancy or longer.

### Example 15

Treatmemt of Climacterium (Peri-Menopausal Syndrome)

To a nonpregnant human female (ca 45 years; 50-80 kg) displaying the signs of menopause or postmenopausal symptoms, including amenorrhea, hot flushes, etc., administer citrulline 0.5 to 20 g per os daily in three divided doses until the symptoms are ameliorated. Thereafter administer 0.5 to 5 g of citrulline daily.

### Example 16

Hormone Replacement Therapy

To a female similar to and displaying the same symptoms as Example 15, administer daily 0.5 to 20 g of citrulline in combination with estrogen (e.g., estradiol valerate) 1-2 mg daily.

### Example 17

Hormone Replacement Therapy

To a female comparable to and displaying the same symptoms as Example 15, administer citrulline 0.5 to 20 g daily with or without one of the following: an estrogen (e.g. estradiol valerate) 1-2 mg daily, or a progestin (e.g. norgestrel, at 150 mg per day). The latter sex steroids to be given either continuously with citrulline, or sequentially - the progestins taken for only 6-12 days per month.

### Example 18:

Hormone Replacement Therapy

To a female comparable to and displaying the same symptoms as Example 16, administer citrulline 0.5 to 20 g daily with or without one of the following, a partial estrogen (e.g. raloxifene) 50-500 mg daily.

The preceding examples can be repeated with similar success by substituting the generically or specifically described reactants and/or operating conditions of this invention for those used in the preceding examples.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of treating or preventing a nitric oxide deficiency syndrome or a disease which can be treated or prevented by increasing endogenous nitric oxide levels in a mammal, comprising
administering to the afflicted mammal an effective amount of
a first agent which enhances the level of endogenous nitric oxide in the target tissue,
optionally, in further combination with a second agent which modulates or enhances nitric oxide synthesis,
with the proviso that the agents are not a natural food source.

2. A method according to claim 1, wherein said agent is citrulline or a citrulline analogue.

3. The method of claim 1, wherein the disease is atherosclerosis, restenosis, hypertension, preeclampsia and/or intrauterine fetal growth retardation.

4. The method of claim 1, wherein the female mammal is a human who has exhibited or is a susceptible to develop preterm labor, early pregnancy loss, infertility or cervical dystocia.

5. The method of claim 1, wherein the female mammal is a human who has exhibited symptoms of climacterium or is a candidate for hormone replacement therapy.

6. The method of claim 1, wherein the disease is altered motility of the intestinal tract, pyloric stenosis or diabetes mellitus.

7. The method of claim 1, wherein the disease is asthma, neonatal respiratory distress syndrome, pulmonary hypertension or adult respiratory distress syndrome.

8. The method of claim 1, wherein the disease is acute inflammation, resistance to infection, SLE-lupus, anaphylactic reaction or allograft rejection.

9. The method of claim 1, wherein the disease is Alzheimer's disease, stroke, growth hormone disorders or behavior changes.

10. The method of claim 1, wherein the modulating agent is L-arginine.

11. The method of claim 1, wherein the mammal is a human and a nitric oxide donor is administered.

12. The method of claim 11, wherein the nitric oxide donor is sodium nitroprusside, nitroglycerin, glyceryltrinitrite, SIN-1, isosorbidmononitrite or isosorbiddinitrite.

13. The method of claim 12, wherein the nitric oxide donor is administered orally.

14. The method of claim 1, wherein the female mammal is a human and the nitric oxide substrate or donor is administered in combination with an estrogen.

15. The method of claim 1, wherein the female mammal is a human who has exhibited symptoms of or is a candidate to develop preterm labor.

16. The method of claim 14, wherein the estrogen is estradiol valerate, conjugated equine estrogens, 17β-estradiol, estrone or estriol.

17. The method of claim 1, wherein the female mammal is a human and the nitric oxide substrate or donor is administered in combination with a progestin.

18. The method of claim 17, wherein the progestin is progesterone, dydrogesterone, medroxyprogesterone, norethisterone, levonorgestrel, drospirenone, or norgestrel.

19. The method of claim 1, wherein the female mammal is a human and the estrogen or progestin are administered continuously.

20. The method of claim 1 wherein the female mammal is a human and estrogen and progesterone are administered sequentially.

21. A pharmaceutical composition comprising an admixture of
(a) citrulline,
(b) a nitric oxide synthesis substrate, a nitric oxide donor or both, and
optionally, one or more of (c) an estrogen and (d) a progestin, in amounts effective to ameliorate the symptoms of an optionally climacterium in a menopausal or postmenopausal female mammal when administered thereto in an amount of estrogen bioequivalent to 1-2 mg of estradiol and an amount of the progestin bioequivalent to 50-300 mg of injected progesterone and an amount of the nitric oxide synthase substrate, nitric oxide donor or both effective to raise the blood level of circulating L-arginine to at least about 10-50 nmolar above the normally 50-100 nmolar circulating levels or raise the nitric oxide donor levels to about 1-1000 nmolar, (e) a cardiovascular agent.

22. A composition of claim 21, wherein (b) is a nitric oxide synthesis substrate.

23. A composition of claim 22, wherein the nitric oxide synthesis substrate (b) is L-arginine.

24. A composition of claim 21, wherein (b) is a nitric oxide donor.

25. A composition of claim 24, wherein the nitric oxide donor is sodium nitroprusside, nitroglycerin, glyceryltrinitrie, SIN-1, isosorbidmononitrite or isosorbiddinitrite.

26. A composition of claim 21, wherein the estrogen (c) is present and is estradiol valerate.

27. A composition of claim 21, wherein the progestin (d) is present and is norgestrel.

28. A composition of claim 21, wherein the cardiovascular agent (e) is propranolol, methyldopa, guanethidine, nifedipine or nicardipine.
